(19) United States
(12) Patent Application Publication
(10) Pub. No.: US 2011/0111527 A1
(43) Pub. Date: May 12, 2011

(54) MULTIMARKER PANEL FOR DIFFERENTIATION OF DILATED CARDIOMYOPATHY AND AS A BASIS FOR DIFFERENTIAL THERAPY

(76) Inventors: Georg Hess, Mainz (DE); Andrea Horsch, Mannheim (DE); Dietmar Zdunek, Tutzing (DE)

(21) Appl. No.: 12/944,874

(22) Filed: Nov. 12, 2010

Related U.S. Application Data
(63) Continuation of application No. PCT/EP2009/055829, filed on May 14, 2009.

(30) Foreign Application Priority Data
May 15, 2008 (EP) 08156235.7
May 29, 2008 (EP) 08157155.6

(51) Int. Cl. G01N 33/68

(52) U.S. Cl. 436/501; 422/68.1

(57) ABSTRACT

The present invention relates to a method for diagnosing if a subject suffering from dilated cardiomyopathy is suffering from ischemic or non-ischemic dilated cardiomyopathy. Further, it relates to a method of determining which medication is to be applied in a subject suffering form after dilated cardiomyopathy. The method includes the steps of determining amounts of troponin, GDF-15, and an angiogenic markers selected from the group of PlGF, endoglin, and sFlt-1 in a sample from the subject and comparing the amounts determined With reference amounts. In one embodiment, the method of the invention further comprises also measuring an amount of a natriuretic peptide.

### RELATED APPLICATIONS

This application is a continuation of PCT/EP2009/055829 filed May 14, 2009 and claims priority to EP 08157155.6 filed May 29, 2008 and EP 08156235.7 filed May 15, 2008.

### FIELD OF THE INVENTION

The present invention relates to diagnostic means and methods. Specifically, the present invention relates to a method for differentiating between the ischemic and non-ischemic form in a subject suffering from dilated cardiomyopathy. Further, it relates to a method of diagnosing to which therapy a subject suffering from dilated cardiomyopathy is susceptible. Finally, the present invention encompasses diagnostic devices and kits for carrying out the aforementioned methods.

### BACKGROUND OF THE INVENTION

Dilated cardiomyopathy (“DCM”) is a syndrome characterized by cardiac enlargement and impaired systolic function of one or both ventricles. The earliest abnormality is usually ventricular enlargement and systolic contractile dysfunction, with the signs and symptoms of congestive heart failure often (but not invariably) developing later. In an occasional patient, the predominant finding is that of contractile dysfunction with only a minimally dilated left ventricle. Apparently normal elite athletes may demonstrate considerable ventricular enlargement with normal systolic performance. It is presumed that this is a physiological adaptation to intense athletic training and does not appear to represent a disease state, although the long-term consequences are not fully known.

The incidence of DCM is reported to be 5 to 8 cases per 100,000 population per year and appears to be increasing, although the true figure is probably higher as a consequence of underreporting of mild or asymptomatic cases.

Although the cause is not definable in many cases, more than 75 specific diseases of heart muscle can produce the clinical manifestations of DCM. It is likely that this condition represents a final common pathway that is the end result of myocardial damage produced by a variety of cytotoxic, metabolic, immunological, familial, and infectious mechanisms.

Dilated cardiomyopathy is a heterogeneous disease. In particular, it is known that an ischemic form of dilated cardiomyopathy exists, as well as a non-ischemic form, a variant of which is non-ischemic cardiomyopathy associated with atherosclerosis. In ischemic dilated cardiomyopathy, a coronary artery disease is regarded as being the underlying cause. In non-ischemic dilated cardiomyopathy, coronary artery disease is not regarded as the principal cause underlying the cardiomyopathy, but genetic, metabolic and inflammatory states instead. Partly, pathological states following hypertrophy in valve diseases or arterial hypertrophy may also cause non-ischemic dilated cardiomyopathy. Non-ischemic cardiomyopathy associated with atherosclerosis is particularly hard to diagnose, as the atherosclerosis is not the cause underlying the cardiomyopathy which is observed.

Dilated cardiomyopathy can easily be diagnosed using echocardiography. Echocardiography, however, does not give information of the cause underlying cardiomyopathy. This holds in particular true in cases when two or more causes are to be taken into consideration, e.g., in diabetes. Moreover, the present methods which are mostly invasive methods cannot describe the mechanism responsible for the progress of the disease.

It is to be understood from the above that it is highly desirable to determine the cause underlying dilated cardiomyopathy in a subject. More preferable, it is desirable to determine if the dilated cardiomyopathy is an ischemic or a non-ischemic dilated cardiomyopathy. Based on such an assessment of the angiogenic status, it can be predicted to which therapy a subject will be susceptible, in order to prevent the severe outcomes of a dilated cardiomyopathy.

Thus, the technical problem underlying the present invention is to be seen as the provision of means and methods for determining in a patient suffering from dilated cardiomyopathy if he or she is suffering from the ischemic or the non-ischemic form thereof, in order to, e.g., select a suitable therapy. The technical problem is solved by the embodiments characterized in the accompanying claims and herein below.

### SUMMARY OF THE INVENTION

Accordingly, the present invention relates to a method for diagnosing if a subject suffering from dilated cardiomyopathy is suffering from ischemic or non-ischemic dilated cardiomyopathy comprising:
a) determining the amounts of the following peptides:
   troponin or a variant thereof; and
   GDF-15 or a variant thereof; and
   one or more angiogenic markers from the group PlGF or a variant thereof, endoglin or a variant thereof and sFlt-1 or a variant thereof
   in a sample of a subject suffering from dilated cardiomyopathy; and
b) comparing the amounts determined in step a) with reference amounts, whereby it is to be diagnosed whether the subject suffers from either ischemic or non-ischemic dilated cardiomyopathy.

In a preferred embodiment, the method of the invention furthermore comprises measuring the amount of a natriuretic peptide.

In other words, the method according to the present invention allows to analyze whether a subject presenting with dilated cardiomyopathy is suffering from the ischemic or the non-ischemic form thereof. As the ischemic form is caused by an underlying coronary artery disease and the non-ischemic form is caused by pathological states other than coronary artery disease, the method of the present invention allows to determine the cause and the mechanism underlying the dilated cardiomyopathy. The method of the present invention may be also used for monitoring, confirmation, and subclassification of dilated cardiomyopathy. The method may be carried out manually or assisted by automation. Preferably, step (a) and/or (b) may in total or in part be assisted by automation, e.g., by a suitable robotic and sensory equipment for the determination in step (a) or a computer-implemented comparison in step (b).

### BRIEF DESCRIPTION OF THE FIGURES

FIG. 1 depicts NT-pro BNP, sensitive troponin T, PlGF, s sFlt-1, endoglin, GDF-15 levels for both patients groups.

FIGS. 2-11 correlate different biomarkers to each other, separately for ischemic and non-ischemic cardiomyopathy.

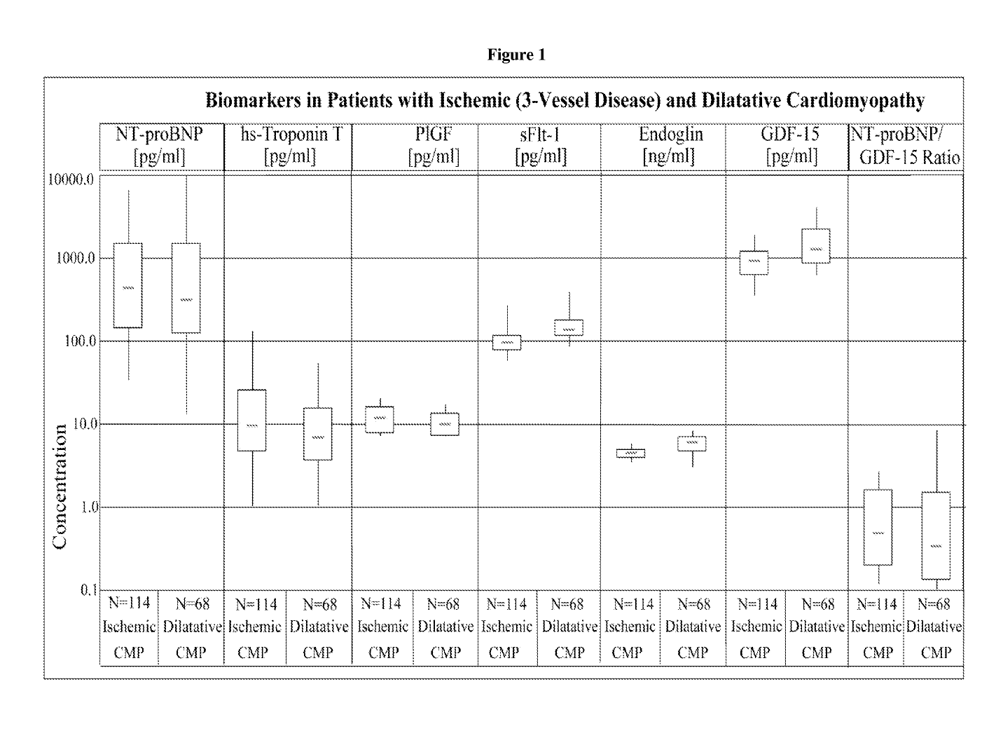

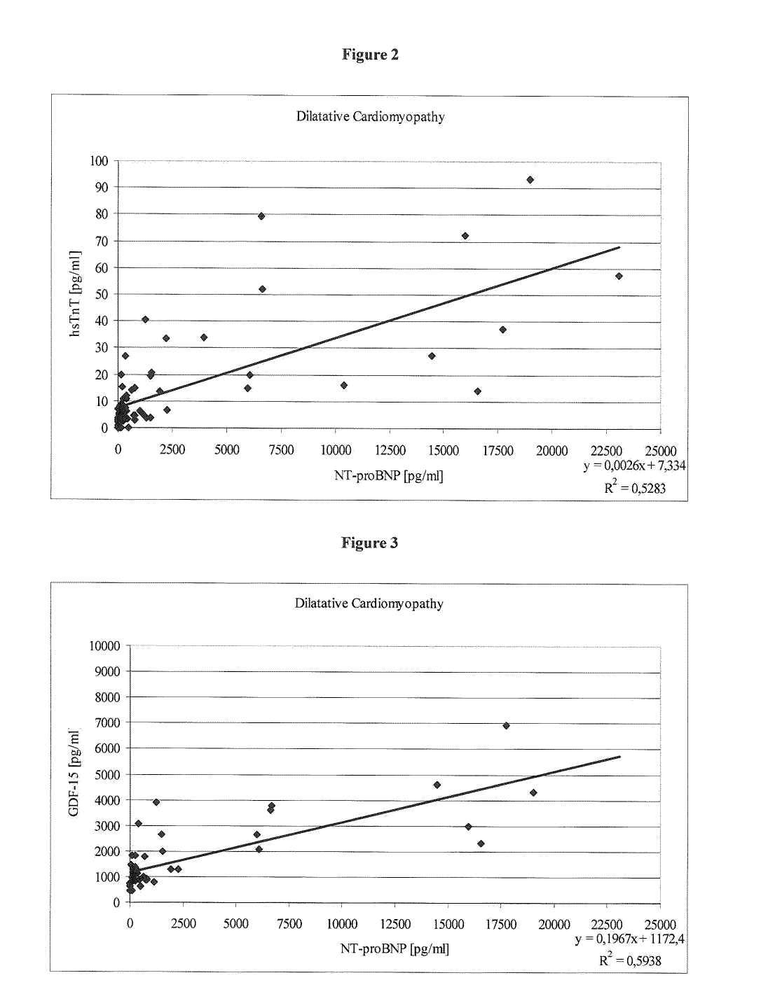

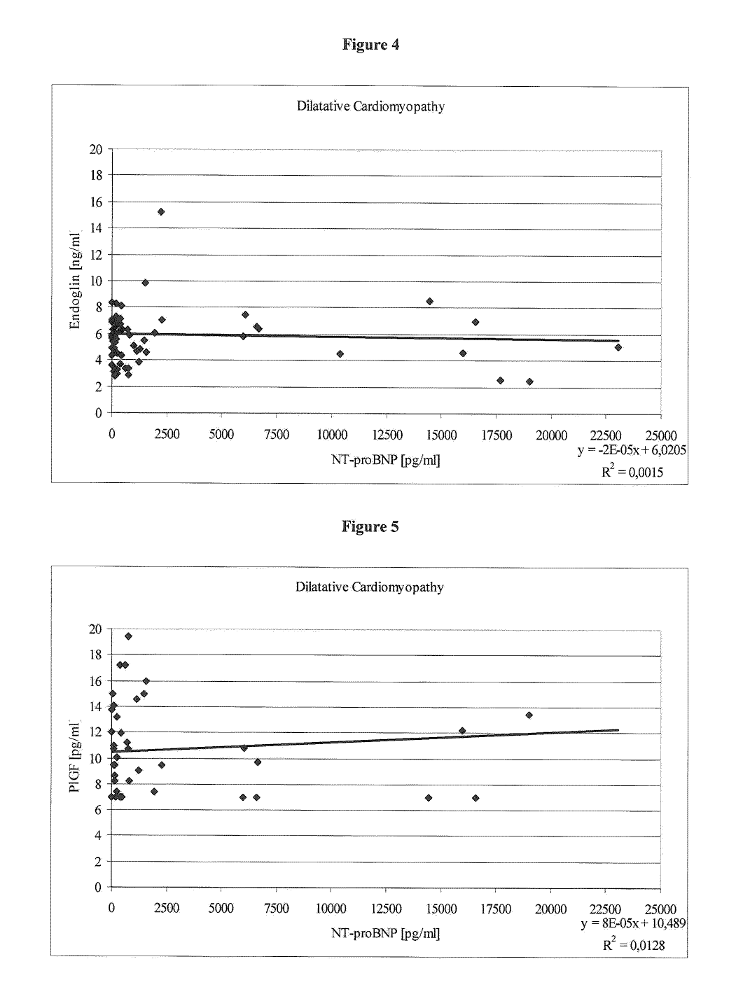

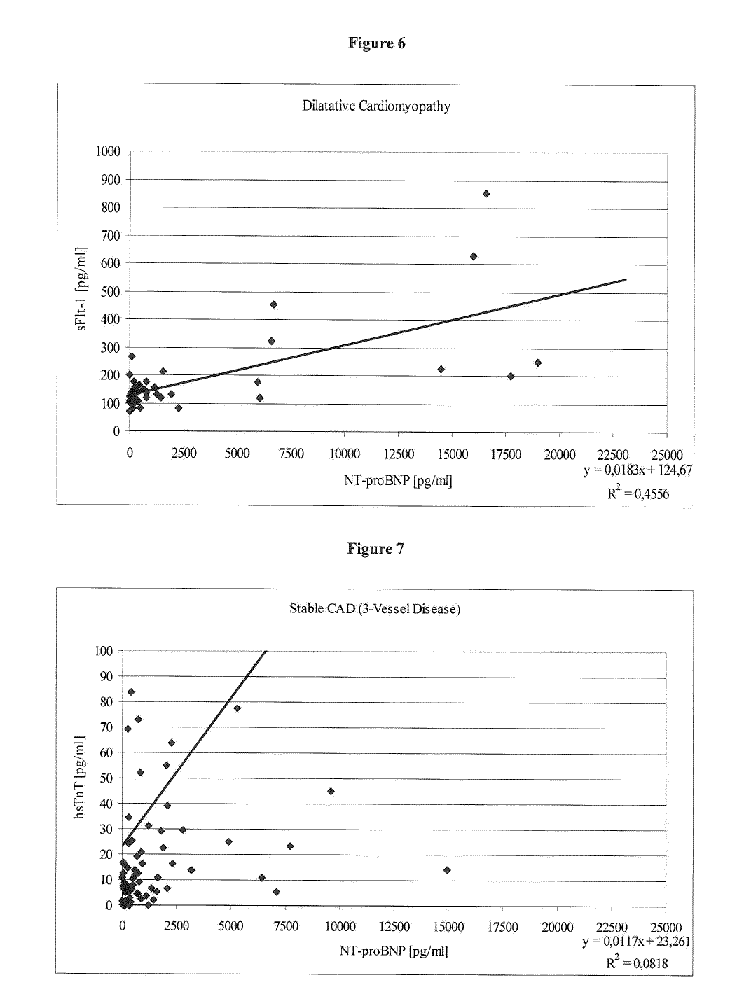

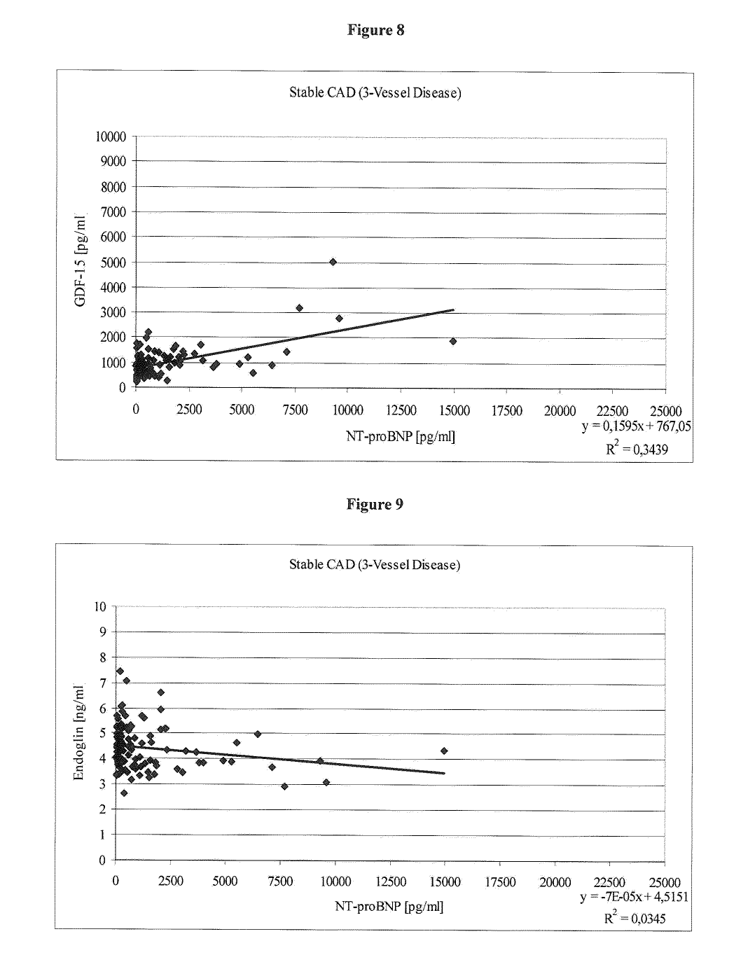

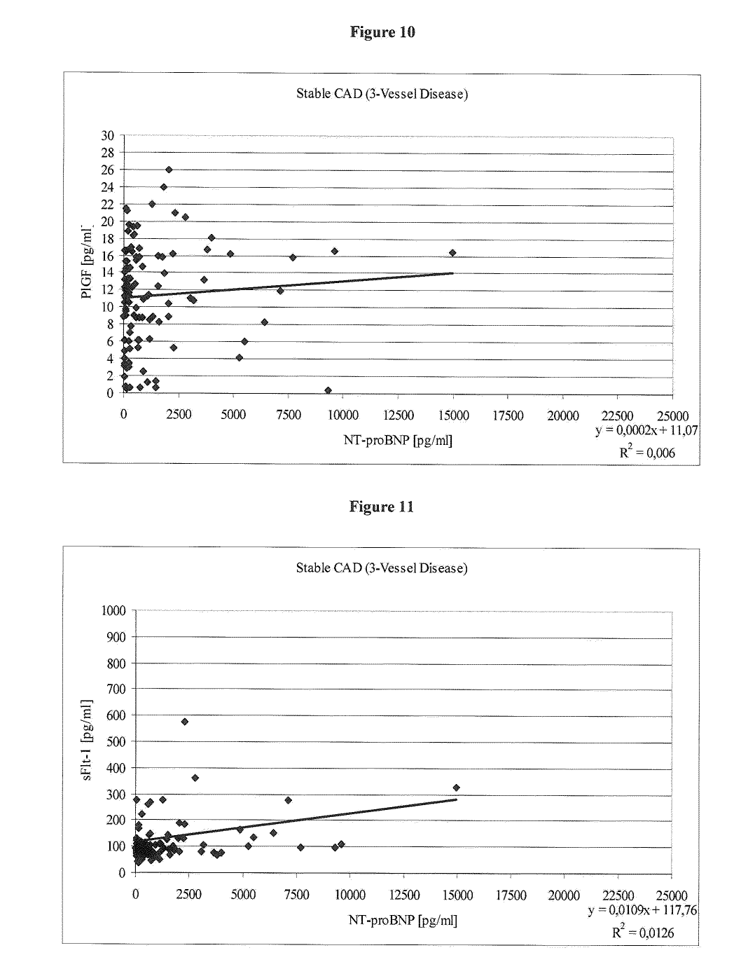

### DETAILED DESCRIPTION OF THE INVENTION

The term “diagnosing” as used herein refers to differentiating between ischemic and non-ischemic dilated cardiomyopathy, i.e. assessing the probability according to which a subject has the ischemic or the non-ischemic form of dilated cardiomyopathy, as referred to in this specification. As will be understood by those skilled in the art, such an assessment is usually not intended to be correct for 100% of the subjects to be diagnosed. The term, however, requires that a statistically significant portion of subjects can be correctly diagnosed to exhibit the said angiogenic status. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the diagnosis will be correct for at least 60%, at least 70%, at least 80%, or at least 90% of the subjects of a given cohort or population.

The method of the present invention, preferably, is an in vitro method. Moreover, it may comprise steps in addition to those explicitly mentioned above. For example, further steps may relate to sample pre-treatments or evaluation of the results obtained by the method.

The term “subject” as used herein relates to animals, preferably mammals, and, more preferably, humans. However, it is envisaged by the present invention that the subject shall be suffering from dilated cardiomyopathy as specified elsewhere herein.

The term “dilated cardiomyopathy” or DCM is known to the person skilled in the art. More specifically, DCM refers to ventricular dilation and impaired contraction (contractile dysfunction) of the left or both ventricles. An individual having DCM often shows symptoms of congestive heart failure CHF.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells may be obtained from the body fluids or the tissues or organs by separating techniques such as centrifugation or cell sorting. Preferably, cell-, tissue- or organ samples are obtained from those cells, tissues or organs which express or produce the peptides referred to herein.

The term “natriuretic peptide” comprises Atrial Natriuretic Peptide (ANP)-type and Brain Natriuretic Peptide (BNP)-type peptides and variants thereof having the same predictive potential. Natriuretic peptides according to the present invention comprise ANP-type and BNP-type peptides and variants thereof (see e.g., Bonow, 1996, Circulation 93: 1946-1950). ANP-type peptides comprise pre-proANP, proANP, NT-proANP, and ANP. BNP-type peptides comprise pre-proBNP, proBNP, NT-proBNP, and BNP. The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP). Preferably, natriuretic peptides according to the present invention are NT-proANP, ANP, and, more preferably, NT-proBNP, BNP, and variants thereof. ANP and BNP are the active hormones and have a shorter half-life than their respective inactive counterparts, NT-proANP and NT-proBNP. BNP is metabolized in the blood, whereas NT-proBNP circulates in the blood as an intact molecule and as such is eliminated renally. The in-vivo half-life of NTproBNP is 120 min longer than that of BNP, which is 20 min (Smith 2000, J Endocrinol. 167: 239-46.). Preanalytics are more robust with NT-proBNP allowing easy transportation of the sample to a central laboratory (Mueller 2004, Clin Chem Lab Med 42: 942-4.). Blood samples can be stored at room temperature for several days or may be mailed or shipped without recovery loss. In contrast, storage of BNP for 48 hours at room temperature or at 4° Celsius leads to a concentration loss of at least 20% (Mueller loc.cit.; Wu 2004, Clin Chem 50: 867-73.). Therefore, depending on the time-course or properties of interest, either measurement of the active or the inactive forms of the natriuretic peptide can be advantageous. The most preferred natriuretic peptides according to the present invention are NT-proBNP or variants thereof. As briefly discussed above, the human NT-proBNP, as referred to in accordance with the present invention, is a polypeptide comprising, preferably, 76 amino acids in length corresponding to the N-terminal portion of the human NT-proBNP molecule. The structure of the human BNP and NT-proBNP has been described already in detail in the prior art, e.g., WO 02/089657, WO 02/083913 or Bonow loc. cit. Preferably, human NT-proBNP as used herein is human NT-proBNP as disclosed in EP 0 648 228 B1. These prior art documents are herewith incorporated by reference with respect to the specific sequences of NT-proBNP and variants thereof disclosed therein. The NT-proBNP referred to in accordance with the present invention further encompasses allelic and other variants of said specific sequence for human NT-proBNP discussed above. Specifically, envisaged are variant polypeptides which are on the amino acid level at least 60% identical, more preferably at least 70%, at least 80%, at least 90%, at least 95%, at least 98% or at least 99% identical, to human NT-proBNP. Substantially similar and also envisaged are proteolytic degradation products which are still recognized by the diagnostic means or by ligands directed against the respective full-length peptide. Also encompassed are variant polypeptides having amino acid deletions, substitutions, and/or additions compared to the amino acid sequence of human NT-proBNP as long as the said polypeptides have NT-proBNP properties. NT-proBNP properties as referred to herein are immunological and/or biological properties. Preferably, the NT-proBNP variants have immunological properties (i.e. epitope composition) comparable to those of NT-proBNP. Thus, the variants shall be recognizable by the aforementioned means or ligands used for determination of the amount of the natriuretic peptides. Biological and/or immunological NT-proBNP properties can be detected by the assay described in Karl et al. (Karl 1999, Scand J Clin Invest 230:177-181), Yeo et al. (Yeo 2003, Clinica Chimica Acta 338:107-115). Variants also include posttranslationally modified peptides such as glycosylated peptides. Further, a variant in accordance with the present invention is also a peptide or polypeptide which has been modified after collection of the sample, for example by covalent or non-covalent attachment of a label, particularly a radioactive or fluorescent label, to the peptide.

The term “cardiac troponin” refers to all troponin isoforms expressed in cells of the heart and, preferably, the subendocardial cells. These isoforms are well characterized in the art as described, e.g., in Anderson 1995, Circulation Research, vol. 76, no. 4: 681-686 and Ferrieres 1998, Clinical Chemistry, 44: 487-493. Preferably, cardiac troponin refers to troponin T and/or troponin I, and, most preferably, to troponin T. It is to be understood that isoforms of troponins may be determined in the method of the present invention together, i.e. simultaneously or sequentially, or individually, i.e. without determining the other isoform at all. Amino acid sequences for human troponin T and human troponin I are disclosed in Anderson, loc cit and Ferrieres 1998, Clinical Chemistry, 44: 487-493.

The term “cardiac troponin” encompasses also variants of the aforementioned specific troponins, i.e., preferably, of troponin I, and more preferably, of troponin T. Such variants have at least the same essential biological and immunological properties as the specific cardiac troponins. In particular, they share the same essential biological and immunological properties if they are detectable by the same specific assays referred to in this specification, e.g., by ELISA Assays using polyclonal or monoclonal antibodies specifically recognizing the said cardiac troponins. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific troponin. Variants may be allelic variants or any other species specific homologs, paralogs, or orthologs. Moreover, the variants referred to herein include fragments of the specific cardiac troponins or the aforementioned types of variants as long as these fragments have the essential immunological and biological properties as referred to above. Such fragments may be, e.g., degradation products of the troponins. Further included are variants which differ due to posttranslational modifications such as phosphorylation or myristylation.

A particularly preferred troponin T assay in the context of the present invention is the ELECSYS 2010 analyzer (Roche Diagnostics) with a detection limit of from 0.001 ng/ml to 0.0015 ng/ml. The term “growth differentiation factor-15” or “GDF-15” relates to a polypeptide being a member of the transforming growth factor (TGF)-β cytokine superfamily The terms polypeptide, peptide and protein are used interchangeable throughout this specification. GDF-15 was originally cloned as macrophage-inhibitory cytokine-1 and later also identified as placental transforming growth factor-β, placental bone morphogenetic protein, non-steroidal anti-inflammatory drug-activated gene-1, and prostate-derived factor (Bootcov loc cit; Hromas, 1997 Biochim Biophys Acta 1354:40-44; Lawton 1997, Gene 203:17-26; Yokoyama-Kobayashi 1997, J Biochem (Tokyo), 122:622-626; Paralkar 1998, J Biol Chem 273:13760-13767). Similar to other TGF-β-related cytokines, GDF-15 is synthesized as an inactive precursor protein, which undergoes disulfide-linked homodimerization. Upon proteolytic cleavage of the N-terminal pro-peptide, GDF-15 is secreted as a ˜28 kDa dimeric protein (Bauskin 2000, Embo J 19:2212-2220). Amino acid sequences for GDF-15 are disclosed in WO99/06445, WO00/70051, WO2005/113585, Bottner 1999, Gene 237: 105-111, Bootcov loc. cit, Tan loc. cit., Baek 2001, Mol Pharmacol 59: 901-908, Hromas loc cit, Paralkar loc cit, Morrish 1996, Placenta 17:431-441 or Yokoyama-Kobayashi loc cit. GDF-15 as used herein encompasses also variants of the aforementioned specific GDF-15 polypeptides. Such variants have at least the same essential biological and immunological properties as the specific GDF-15 polypeptides. In particular, they share the same essential biological and immunological properties if they are detectable by the same specific assays referred to in this specification, e.g., by ELISA assays using polyclonal or monoclonal antibodies specifically recognizing the said GDF-15 polypeptides. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific GDF-15 polypeptides. Moreover, the variants referred to herein include fragments of the specific GDF-15 polypeptides or the aforementioned types of variants as long as these fragments have the essential immunological and biological properties as referred to above. Such fragments may be, e.g., degradation products of the GDF-15 polypeptides. Further included are variants which differ due to posttranslational modifications such as phosphorylation or myristylation. A preferred GDF-15 assay in the context of the present invention is the assay as described by Wollert et al. in Clinical Chemistry 53, No 2, 2007, p. 284-291.

The term “PlGF (placental growth factor)” as used herein refers to a placenta derived growth factor which is a 149-amino-acid-long polypeptide and is highly homologous (53% identity) to the platelet-derived growth factor-like region of human vascular endothelial growth factor (VEGF). Like VEGF, PlGF has angiogenic activity in vitro and in vivo. For example, biochemical and functional characterization of PlGF derived from transfected COS-1 cells revealed that it is a glycosylated dimeric secreted protein able to stimulate endothelial cell growth in vitro (Maqlione1993, Oncogene 8(4):925-31). Preferably, PlGF refers to human PlGF, more preferably, to human PlGF having an amino acid sequence as shown in Genebank accession number P49763, GI: 17380553. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific PlGF. Variants may be allelic variants, splice variants or any other species specific homologs, paralogs, or orthologs. Moreover, the variants referred to herein include fragments of the specific PlGF or the aforementioned types of variants as long as these fragments have the essential immunological and biological properties as referred to above. Such fragments may be, e.g., degradation products of PlGF. Further included are variants which differ due to posttranslational modifications such as phosphorylation or myristylation.

The term “endoglin” as used herein refers to a polypeptide having a molecular weight of 180 kDa non-reduced, 95 kDa after reduction and 66 kDa in its reduced and N-deglycosylated form. The polypeptide is capable of forming dimers and bins to TGF-β and TGF-β receptors (see below). Endoglin may be phosphorylated. Preferably, endoglin refers to human endoglin. More preferably, human endoglin has an amino acid sequence as shown in Genebank accession number AAC63386.1, GI: 3201489. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific endoglin. Variants may be allelic variants, splice variants or any other species specific homologs, paralogs, or orthologs. Moreover, the variants referred to herein include fragments of the specific endoglin or the aforementioned types of variants as long as these fragments have the essential immunological and biological properties as referred to above. Such fragments may be, e.g., degradation products of endoglin. Further included are variants which differ due to posttranslational modifications such as phosphorylation or myristylation.

The term “soluble (s)Flt-1” as used herein refers to polypeptide which is a soluble form of the VEGF receptor FLT1. It was identified in conditioned culture medium of human umbilical vein endothelial cells. The endogenous soluble Flt1 (sFlt-1) receptor is chromatographically and immunologically similar to recombinant human sFlt-1 and binds [1251] VEGF with a comparable high affinity. Human sFlt-1 is shown to form a VEGF-stabilized complex with the extracellular domain of KDR/Flk-1 in vitro. Preferably, sFlt-1 refers to human sFlt-1. More preferably, human sFlt-1 can be deduced from the amino acid sequence of Flt-1 as shown in Genebank accession number P17948, GI: 125361. An amino acid sequence for mouse sFlt-1 is shown in Genebank accession number BAA24499.1, GI: 2809071. Moreover, it is to be understood that a variant as referred to in accordance with the present invention shall have an amino acid sequence which differs due to at least one amino acid substitution, deletion and/or addition wherein the amino acid sequence of the variant is still, preferably, at least 50%, 60%, 70%, 80%, 85%, 90%, 92%, 95%, 97%, 98%, or 99% identical with the amino sequence of the specific sFlt-1. Variants may be allelic variants, splice variants or any other species specific homologs, paralogs, or orthologs. Moreover, the variants referred to herein include fragments of the specific sFlt-1 or the aforementioned types of variants as long as these fragments have the essential immunological and biological properties as referred to above. Such fragments may be, e.g., degradation products of sFlt-1. Further included are variants which differ due to posttranslational modifications such as phosphorylation or myristylation.

In the context of the present invention, PlGF, endoglin and sFlt-1 will be referred to as “angiogenic markers”.

Determining the amount of the peptides or polypeptides referred to in this specification relates to measuring the amount or concentration, preferably semi-quantitatively or quantitatively. Measuring can be done directly or indirectly. Direct measuring relates to measuring the amount or concentration of the peptide or polypeptide based on a signal which is obtained from the peptide or polypeptide itself and the intensity of which directly correlates with the number of molecules of the peptide present in the sample. Such a signal—sometimes referred to herein as intensity signal—may be obtained, e.g., by measuring an intensity value of a specific physical or chemical property of the peptide or polypeptide. Indirect measuring includes measuring of a signal obtained from a secondary component (i.e. a component not being the peptide or polypeptide itself) or a biological read out system, e.g., measurable cellular responses, ligands, labels, or enzymatic reaction products.

In accordance with the present invention, determining the amount of a peptide or polypeptide can be achieved by all known means for determining the amount of a peptide in a sample. Said means comprise immunoassay devices and methods which may utilize labeled molecules in various sandwich, competition, or other assay formats. Said assays will develop a signal which is indicative for the presence or absence of the peptide or polypeptide. Moreover, the signal strength can, preferably, be correlated directly or indirectly (e.g., reverse-proportional) to the amount of polypeptide present in a sample. Further suitable methods comprise measuring a physical or chemical property specific for the peptide or polypeptide such as its precise molecular mass or NMR spectrum. Said methods comprise, preferably, biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays (available for example on ELECSYS analyzers), CBA (an enzymatic Cobalt Binding Assay, available for example on Roche-Hitachi analyzers), and latex agglutination assays (available for example on Roche-Hitachi analyzers).

Preferably, determining the amount of a peptide or polypeptide comprises the steps of (a) contacting a cell capable of eliciting a cellular response the intensity of which is indicative of the amount of the peptide or polypeptide with the said peptide or polypeptide for an adequate period of time, (b) measuring the cellular response. For measuring cellular responses, the sample or processed sample is, preferably, added to a cell culture and an internal or external cellular response is measured. The cellular response may include the measurable expression of a reporter gene or the secretion of a substance, e.g., a peptide, polypeptide, or a small molecule. The expression or substance shall generate an intensity signal which correlates to the amount of the peptide or polypeptide.

Also preferably, determining the amount of a peptide or polypeptide comprises the step of measuring a specific intensity signal obtainable from the peptide or polypeptide in the sample. As described above, such a signal may be the signal intensity observed at a mass to charge (m/z) variable specific for the peptide or polypeptide observed in mass spectra or a NMR spectrum specific for the peptide or polypeptide.

Determining the amount of a peptide or polypeptide may, preferably, comprise the steps of (a) contacting the peptide with a specific ligand, (b) (optionally) removing non-bound ligand, (c) measuring the amount of bound ligand. The bound ligand will generate an intensity signal. Binding according to the present invention includes both covalent and non-covalent binding. A ligand according to the present invention can be any compound, e.g., a peptide, polypeptide, nucleic acid, or small molecule, binding to the peptide or polypeptide described herein. Preferred ligands include antibodies, nucleic acids, peptides or polypeptides such as receptors or binding partners for the peptide or polypeptide and fragments thereof comprising the binding domains for the peptides, and aptamers, e.g., nucleic acid or peptide aptamers. Methods to prepare such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g., phage display. Antibodies as referred to herein include both polyclonal and monoclonal antibodies, as well as fragments thereof, such as Fv, Fab and F(ab)2 fragments that are capable of binding antigen or hapten. The present invention also includes single chain antibodies and humanized hybrid antibodies wherein amino acid sequences of a non-human donor antibody exhibiting a desired antigen-specificity are combined with sequences of a human acceptor antibody. The donor sequences will usually include at least the antigen-binding amino acid residues of the donor but may comprise other structurally and/or functionally relevant amino acid residues of the donor antibody as well. Such hybrids can be prepared by several methods well known in the art. Preferably, the ligand or agent binds specifically to the peptide or polypeptide. Specific binding according to the present invention means that the ligand or agent should not bind substantially to (“cross-react” with) another peptide, polypeptide or substance present in the sample to be analyzed. Preferably, the specifically bound peptide or polypeptide should be bound with at least 3 times higher, more preferably at least 10 times higher and even more preferably at least 50 times higher affinity than any other relevant peptide or polypeptide. Non-specific binding may be tolerable, if it can still be distinguished and measured unequivocally, e.g., according to its size on a Western Blot, or by its relatively higher abundance in the sample. Binding of the ligand can be measured by any method known in the art. Preferably, said method is semi-quantitative or quantitative. Suitable methods are described in the following.

First, binding of a ligand may be measured directly, e.g., by NMR or surface plasmon resonance.

Second, if the ligand also serves as a substrate of an enzymatic activity of the peptide or polypeptide of interest, an enzymatic reaction product may be measured (e.g., the amount of a protease can be measured by measuring the amount of cleaved substrate, e.g., on a Western Blot). Alternatively, the ligand may exhibit enzymatic properties itself and the “ligand/peptide or polypeptide” complex or the ligand which was bound by the peptide or polypeptide, respectively, may be contacted with a suitable substrate allowing detection by the generation of an intensity signal. For measurement of enzymatic reaction products, preferably the amount of substrate is saturating. The substrate may also be labeled with a detectable label prior to the reaction. Preferably, the sample is contacted with the substrate for an adequate period of time. An adequate period of time refers to the time necessary for an detectable, preferably measurable, amount of product to be produced, Instead of measuring the amount of product, the time necessary for appearance of a given (e.g., detectable) amount of product can be measured.

Third, the ligand may be coupled covalently or non-covalently to a label allowing detection and measurement of the ligand. Labeling may be done by direct or indirect methods. Direct labeling involves coupling of the label directly (covalently or non-covalently) to the ligand. Indirect labeling involves binding (covalently or non-covalently) of a secondary ligand to the first ligand. The secondary ligand should specifically bind to the first ligand. Said secondary ligand may be coupled with a suitable label and/or be the target (receptor) of tertiary ligand binding to the secondary ligand. The use of secondary, tertiary or even higher order ligands is often used to increase the signal. Suitable secondary and higher order ligands may include antibodies, secondary antibodies, and the well-known streptavidin-biotin system (Vector Laboratories, Inc.). The ligand or substrate may also be “tagged” with one or more tags as known in the art. Such tags may then be targets for higher order ligands. Suitable tags include biotin, digoxigenin, His-Tag, Glutathione-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium, enzymatically active labels, radioactive labels, magnetic labels (e.g., magnetic beads, including paramagnetic and superparamagnetic labels), and fluorescent labels. Enzymatically active labels include e.g., horseradish peroxidase, alkaline phosphatase, beta-galactosidase, luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star (Amersham Biosciences), ECF (Amersham Biosciences). A suitable enzyme-substrate combination may result in a colored reaction product, fluorescence or chemoluminescence, which can be measured according to methods known in the art (e.g., using a light-sensitive film or a suitable camera system). As for measuring the enzymatic reaction, the criteria given above apply analogously. Typical fluorescent labels include fluorescent proteins (such as GFP and its derivatives), Cy3, Cy5, Texas Red, fluorescein, and the Alexa dyes (e.g., Alexa 568). Further fluorescent labels are available e.g., from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g., a light-sensitive film or a phosphor imager. Suitable measurement methods according the present invention also include precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoroimmunoassay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamide gel electrophoresis (SDS-PAGE), Western Blotting, and mass spectrometry), can be used alone or in combination with labeling or other detection methods as described above.

The amount of a peptide or polypeptide may be, also preferably, determined as follows: (a) contacting a solid support comprising a ligand for the peptide or polypeptide as specified above with a sample comprising the peptide or polypeptide and (b) measuring the amount peptide or polypeptide which is bound to the support. The ligand, preferably chosen from the group consisting of nucleic acids, peptides, polypeptides, antibodies and aptamers, is preferably present on a solid support in immobilized form. Materials for manufacturing solid supports are well known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The ligand or agent may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. Suitable methods for fixing/immobilizing said ligand are well known and include, but are not limited to ionic, hydrophobic, covalent interactions and the like. It is also contemplated to use “suspension arrays” as arrays according to the present invention (Nolan 2002, Trends Biotechnol. 20(1):9-12). In such suspension arrays, the carrier, e.g., a microbead or microsphere, is present in suspension. The array consists of different microbeads or microspheres, possibly labeled, carrying different ligands. Methods of producing such arrays, for example based on solid-phase chemistry and photo-labile protective groups, are generally known (U.S. Pat. No. 5,744,305).

The term “amount” as used herein encompasses the absolute amount of a polypeptide or peptide, the relative amount or concentration of the said polypeptide or peptide as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response levels determined from biological read out systems in response to the peptides or intensity signals obtained from specifically bound ligands. It is to be understood that values correlating to the aforementioned amounts or parameters can also be obtained by all standard mathematical operations.

The term “comparing” as used herein encompasses comparing the amount of the peptide or polypeptide comprised by the sample to be analyzed with an amount of a suitable reference source specified elsewhere in this description. It is to be understood that comparing as used herein refers to a comparison of corresponding parameters or values, e.g., an absolute amount is compared to an absolute reference amount while a concentration is compared to a reference concentration or an intensity signal obtained from a test sample is compared to the same type of intensity signal of a reference sample. The comparison referred to in step (b) of the method of the present invention may be carried out manually or computer assisted. For a computer assisted comparison, the value of the determined amount may be compared to values corresponding to suitable references which are stored in a database by a computer program. The computer program may further evaluate the result of the comparison, i.e. automatically provide the desired assessment in a suitable output format. Based on the comparison of the amount determined in step a) and the reference amount, it is possible to identify the cause of dilated cardiomyopathy. Therefore, the reference amount is to be chosen so that either a difference or a similarity in the compared amounts allows identifying those subjects which have ischemic dilated cardiomyopathy and those having non-ischemic dilated cardiomyopathy.

Accordingly, the term “reference amounts” as used herein refers to amounts of the polypeptides which allow allocating the dilated cardiomyopathy of a subject as either ischemic or non-ischemic. Therefore, the reference may either be derived from (i) a subject known to have ischemic dilated cardiomyopathy or (ii) a subject known to have a non-ischemic dilated cardiomyopathy. Moreover, the reference amounts, preferably, define thresholds. Suitable reference amounts or threshold amounts may be determined by the method of the present invention from a reference sample to be analyzed together, i.e. simultaneously or subsequently, with the test sample. A reference amount serving as a threshold may be derived from the upper limit of normal (ULN), i.e. the upper limit of the physiological amount to be found in a population of subjects (e.g., patients enrolled for a clinical trial). The ULN for a given population of subjects can be determined by various well known techniques. A suitable technique may be to determine the median of the population for the peptide or polypeptide amounts to be determined in the method of the present invention.

Angiogenesis is known as the formation of new blood vessels from already existing blood vessels by a capillary sprouting process. The process is under physiological conditions essentially driven by angiogenic growth factors such as the vascular endothelial growth factor (VEGF). The expression of such angiogenic growth factors is regulated pivotally by hypoxia. Thus, if a tissue becomes ischemic, the cells will start to produce angiogenic growth factors which will attract new blood vessels to the tissue by angiogenesis.

However, the capability of a subject for angiogenesis, i.e. its angiogenic status, is dependent on complex biological parameters. Various angiogenesis promoting factors as well as inhibitors of angiogenesis have been reported (Nyberg 2005, Cancer Res 65:3967-3979).

Angiogenesis is observed during tumor growth where the growing tumor becomes more and more affected by hypoxia.

Other disease conditions which are accompanied by hypoxia and ischemia include the coronary artery diseases. Said diseases are characterized by stenosis or occlusion of vessels of the coronary artery system, e.g., by atherosclerosis or thromboembolic occlusions. Coronary artery diseases result in ischemia of the myocardium. Said ischemia, if left untreated, may severely interfere with the physiological function of the heart and result in cardiac disorders including heart failure or even myocardial infarction. For patients suffering from coronary artery diseases, an angiogenic therapy may assist in avoiding the aforementioned life-threatening conditions. Moreover, angiogenic therapies may even help to circumvent complicated cardiac interventions such as stent implantation or bypass surgery.

As set forth above already, various factors besides VEGF have been reported to play a role in angiogenesis. Placental growth factor (PlGF) is a closely related growth factor suggested to play a role in the related process of arteriogenesis together with its putative receptor Flt-1 (Khurana 2005, Circulation 111:2828-2836). Other factors which are possibly involved in arteriogenesis and angiogenesis are the members of the Transforming growth factor-beta superfamily as well as their receptors or binding partners such as the ALK receptors or endoglin (van Laake 2006, Circulation, 114:2288-2297; Bobik 2006, Arterioscler Thromb Vasc Biol 26: 1712-1720; Bertolino 2005, Chest Supplement 128: 585-590). Fibroblast growth factor (FGF), Platlet derived growth factor (PDGF) as well as cytokines and matrix-metalloproteinases have been also described as potent angiogenic factors (Nyberg, loc.cit.).

Patients suffering from myocardial infarction MI can be diagnosed using cardiac troponins, preferably troponin T or I, most preferably troponin T. Myocardial infarction is regarded as being caused by a necrotic state of the myocard, i.e., cell death. Cardiac troponins are released following cell death and can hence be used for the diagnosis of MI. If the amount of troponin T in the blood is elevated, i.e. above 0.1 ng/ml, an acute cardiovascular event is assumed and the patent is treated accordingly. However, it is known that cardiac troponins are also be released (in small amounts) in pathological states preceding cell death, e.g., ischemia. Preferably, the amount of a cardiac troponin, particularly troponin, is determined with a very sensitive troponin t test system in order to allow a reliable determination of very low cardiac troponin amounts, preferably said test system is capable of determining amounts of 0.002 ng/ml troponin in a sample, preferably, in a blood, blood serum or blood plasma sample. A particularly preferred troponin T assay in the context of the present invention is the ELECSYS 2010 analyzer (Roche Diagnostics GmbH) with a detection limit of from 0.001 ng/ml to 0.0015 ng/ml, in general 0.0015 ng/ml.

Heart failure is a condition that can result from any structural or functional cardiac disorder that impairs the ability of the heart to fill with or pump a sufficient amount of blood throughout the body. Even with the best therapy, heart failure is associated with an annual mortality of about 10%. Heart failure is a chronic disease; it can, inter alia, occur either following an acute cardiovascular event (like myocardial infarction), or it can occur, e.g., as a consequence of inflammatory or degenerative changes in myocardial tissue. Heart failure patients are classified according to the NYHA system in classes I, II, III, and IV. A patient having heart failure will not be able to fully restore his health without receiving a therapeutic treatment.

Myocardial dysfunction is a general term, describing several pathological states of the heart muscle (myocard). A myocardial dysfunction may be a temporary pathological state (caused by, e.g., ischemia, toxic substances, alcohol, . . . ), contrary to heart failure. Myocardial dysfunction may disappear after removing the underlying cause. A symptomless myocardial dysfunction may, however, also develop into heart failure (which has to be treated in a therapy). A myocardial dysfunction may, however, also be a heart failure, a chronic heart failure, even a severe chronic heart failure.

Myocardial dysfunction and heart failure often remain undiagnosed, particularly when the condition is considered “mild.” The conventional diagnostic techniques for heart failure are based on the well known vascular volume stress marker NT-proBNP. However, the diagnosis of heart failure under some medical circumstances based on NT-proBNP appears to be incorrect for a significant number of patients but not all (e.g., Beck 2004, Canadian Journal of Cardiology 20: 1245-1248; Tsuchida 2004, Journal of Cardiology, 44:1-11). However, especially patients which suffer from heart failure would urgently need a supportive therapy of heart failure. On the other hand, as a consequence of an incorrect diagnosis of heart failure, many patients will receive a treatment regimen which is insufficient or which may have even adverse side effects.

In the context of the present invention, the following reference values are considered indicative for the presence of ischemic or non-ischemic cardiomyopathy.

Cardiac troponin, preferably troponin I or troponin T, in particular troponin T referred to herein: preferably 0.008 ng/ml, more preferably 0.005 ng/ml, most preferably 0.003 ng/ml.

The person skilled in the art knows that the amounts measured with a specific test (here: ELECSYS 2010 by Roche Diagnostics) may change within the limits indicated in the instructions and information for use. The instructions and information for use are herewith incorporated by reference.

GDF-15 referred to herein: preferably 1000 pg/ml, more preferably 750 pg/ml, most preferably 600 pg/ml.

PlGF referred to herein: preferably 11 pg/ml, more preferably 8 pg/ml. Endoglin referred to herein: preferably 4.8 ng/ml, more preferably 4.2 ng/ml. sFlt-1 referred to herein: preferably 142 pg/ml, more preferably 120 pg/ml.

When the amounts of the cardiac troponin (troponin T or I) measured in an individual are above those cited beforehand, this is indicative that the individual suffers from ischemic DCM (i.e. he will likely not suffer from non-ischemic DCM). When the amounts are below the reference values, the inverse holds true.

Also, in accordance with the present invention, and with respect to the reference values cited beforehand, an increased amount of cardiac troponin, in particular troponin T is indicative for myocardial ischemia and hypoxia and/or necrosis, whereas with respect to the reference values, a decreased amount of cardiac troponin, in particular troponin T is indicative for the absence of myocardial ischemia and hypoxia and/or necrosis. Thus, in a preferred embodiment of the method of the present invention, an increased amount of cardiac troponin, in particular troponin T is indicative for myocardial ischemia and hypoxia and/or necrosis.

When the amounts of GDF-15 measured in an individual are above those cited beforehand, this is indicative that the individual suffers from non-ischemic DCM (i.e. he will likely not suffer from ischemic DCM). When the amounts are below those values, the inverse holds true.

Also, in accordance with the present invention, and with respect to the reference values cited beforehand, an increased amount of GDF-15 is indicative for inflammatory processes occurring in the myocard, whereas with respect to the reference values a decreased amount of GDF-15 is indicative for the absence of inflammatory processes in the myocard. Thus, in a preferred embodiment of the method of the present invention, an increased amount of GDF-15 is indicative for inflammatory processes occurring in the myocard, whereas a decreased amount of GDF-15 is indicative for the absence of inflammatory processes in the myocard.

Moreover, it has been found that each of said biomarkers is statistically independent from each other

In accordance with the present invention, and with respect to the above-cited reference values, an increased amount of endoglin and sFlt-1 and a decreased amount of PlGF are indicative for non-ischemic cardiomyopathy, whereas with respect to the reference values, a decreased amount of endoglin and sFlt-1 and an increased amount of PlGF are indicative for a pro-angiogenic status. An angiogenic (“pro-angiogenic”) status is indicative for the occurrence of ischemic states or processes, whereas an anti-angiogenic status is indicative for the non-occurrence of ischemic states or processes. Thus, in a preferred embodiment of the method of the present invention, an increased amount of endoglin and sFlt-1 and a decreased amount of PlGF are indicative for a non-ischemic cardiomyopathy. In another preferred embodiment of the method of the present invention, a decreased amount of endoglin and sFlt-1 and an increased amount of PlGF are indicative for an ischemic cardiomyopathy in the respective individual. In one embodiment of the invention, both PlGF and sFlt-1 are measured. In a further embodiment of the present invention, only one of PlGF and sFlt-1 is measured, preferably PlGF.

In individuals suffering from non-ischemic cardiomyopathy associated with atherosclerosis, the reference values are the same or similar to those cited beforehand, with the exception of endoglin. One benefit of the present invention is that it is possible to diagnose if an individuum suffers from non-ischemic DCM associated with atherosclerosis. In these cases, the endoglin values are as follows: preferably 4.8 ng/ml, more preferably 4.2 ng/ml, in particular 3.8 ng/ml.

Natriuretic peptide, preferably BNP or NT-proBNP, in particular NT-proBNP as referred to herein: preferably 125 pg/ml, in particular 200 pg/ml, most preferably 350 pg/ml.

Furthermore, in accordance with a preferred embodiment of the present invention, and with respect to the above-cited reference values, an increased amount of natriuretic peptide, in particular NT-proBNP is indicative for myocardial dysfunction, in particular heart failure, whereas with respect to the reference values, a decreased amount of natriuretic peptide, in particular NT-proBNP is indicative for the absence of myocardial dysfunction, in particular for the absence of heart failure. Thus, in a preferred embodiment of the method of the present invention, an increased amount of natriuretic peptide, in particular NT-proBNP is indicative for myocardial dysfunction, in particular heart failure. In another preferred embodiment of the method of the present invention, a decreased amount of natriuretic peptide, in particular NT-proBNP is indicative for the absence of myocardial dysfunction, in particular for the absence of heart failure.

The occurrence of myocardial dysfunction or heart failure is independent of the fact whether the individual suffers from ischemic or non-ischemic dilated cardiomyopathy. Both forms of cardiomyopathy can be accompanied by more or less severe forms of myocardial dysfunction or heart failure. Both terms are known to the person skilled in the art.

The present invention therefore also relates to cardiac disorders, preferably from the group myocardial dysfunction and heart failure.

The term “myocardial dysfunction” as used herein is a general term and relates to several pathological states of the myocard. A myocardial dysfunction may be a temporary pathological state (caused by e.g., ischemia, toxic substances, alcohol, . . . ). Myocardial dysfunction may disappear after removing the underlying cause. In the context of the present invention, the myocardial dysfunction can be a symptomless myocardial dysfunction. A myocardial dysfunction, in particular a symptomless myocardial dysfunction, may also develop into heart failure. A myocardial dysfunction may also be a severe chronic heart failure. In general, a myocardial dysfunction is an impaired systolic and/or diastolic function of the heart, and a myocardial dysfunction may occur with or without heart failure. Any heart failure mentioned beforehand my be symptomless.

The term “heart failure” as used herein relates to an impaired systolic and/or diastolic function of the heart. Preferably, heart failure referred to herein is also chronic heart failure. Heart failure can be classified into a functional classification system according to the New York Heart Association (NYHA). Patients of NYHA Class I have no obvious symptoms of cardiovascular disease but already have objective evidence of functional impairment. Physical activity is not limited, and ordinary physical activity does not cause undue fatigue, palpitation, or dyspnea (shortness of breath). Patients of NYHA class II have slight limitation of physical activity. They are comfortable at rest, but ordinary physical activity results in fatigue, palpitation, or dyspnea. Patients of NYHA class III show a marked limitation of physical activity. They are comfortable at rest, but less than ordinary activity causes fatigue, palpitation, or dyspnea. Patients of NYHA class IV are unable to carry out any physical activity without discomfort. They show symptoms of cardiac insufficiency at rest. Heart failure, i.e., an impaired systolic and/or diastolic function of the heart, can be determined also by, for example, echocardiography, angiography, szintigraphy, or magnetic resonance imaging. This functional impairment can be accompanied by symptoms of heart failure as outlined above (NYHA class II-IV), although some patients may present without significant symptoms (NYHA I). Moreover, heart failure is also apparent by a reduced left ventricular ejection fraction (LVEF). More preferably, heart failure as used herein is accompanied by a left ventricular ejection fraction (LVEF) of less than 60%, of 40% to 60% or of less than 40%.

Accordingly, determining the amount of a natriuretic peptide furthermore permits to assess whether the individual suffering from ischemic or non-ischemic dilated cardiomyopathy is also suffering from myocardial dysfunction and/or heart failure, and if the dysfunction/failure is severe or less severe.

Accordingly, the method of the present invention provides for a highly reliable diagnosis. The techniques which are currently used to resolve this issue are time consuming and cost intensive. The method of the present invention, however, allows a reliable, fast and less cost intensive diagnosis and can be implemented even in portable assays, such as test strips. Therefore, the method is particularly well suited for diagnosing emergency patients. Thanks to the findings of the present invention, a suitable angiogenic therapy for a subject can be reliably selected. Severe side effects caused by the wrong treatment of patients can be avoided.

The present invention also relates to a method of determining and/or deciding which medication is to be applied in a subject suffering from after dilated cardiomyopathy, said method comprising
a) determining the amounts of the following peptides:
   troponin or a variant thereof; and
   GDF-15 or a variant thereof;
   one or more angiogenic markers from the group PlGF or a variant thereof; endoglin or a variant thereof, and sFlt-1 or a variant thereof;
   optionally, a natriuretic peptide or a variant thereof;
   in a sample of a subject suffering from dilated cardiomyopathy; and
b) comparing the amounts determined in step a) with reference amounts, whereby it is to be diagnosed whether the subject suffers from either ischemic or non-ischemic dilated cardiomyopathy.
c) determining and/or deciding, in accordance with the amounts determined in a) and/or the information on the form of dilated cardiomyopathy obtained in b), which medication is to be applied to the subject.

The medication is selected from the following:

A) Agents affecting cardiac function, preferably: beta blockers like proprenolol, metoprolol, bisoprolol, carvedilol, bucindolol, nebivolol; nitrates; adrenergic agonists, like dobutamine, dopamine, epinephrine, isoprotenerol, norepinephrine, phenylephrine; positive inotropic agents, like digoxin, digitoxin; diuretics, in particular loop diuretics, thiazide and thiazide-like diuretics, K-sparing diuretics, type I mineralocorticoid receptor antagonists, carbonic anhydrase inhibitors. vasopressure antagonists.

The information whether these agents should be administered is provided if an elevated level of a natriuretic peptide is measured. Suitable natriuretic peptides are BNP, NT-proBNP, ANP, NT-proANP; preferably BNP or NT-proBNP, in particular NT-proBNP.

When a level of natriuretic peptide of, in the case of NT-proBNP, >300 pg/ml, preferably >500 pg/ml, more preferably >800 pg/ml, still more preferably >2000 pg/ml is reached, one or more of the above-cited drugs should be administered.

B) anti-inflammatory drugs, preferably: ACE inhibitors, in particular Enalapril, Captopril, Ramipril, Trandolapril; angiotensin receptor antagonists and aldosterone antagonists, in particular Losartan, Valsartan, Irbesartan, Candesartan, Telmisartan, Eprosartan, Spironolactone; statins, in particular Atorvastatin, Fluvastatin, Lovastatin, Pravastatin, Rosuvastatin, Simvastatin; NSAIDS; selective COX-2 inhibitors

The information whether these agents should be administered is provided if an elevated level of GDF-15 which is indicative for inflammatory processes is measured.

When a level of GDF-15 of >800 pg/ml, preferably >1200 pg/ml, more preferably >1500 pg/ml, in particular >2000 pg/ml is reached, one or more of the above-cited drugs should be administered.

C) Medicaments for a pro-angiogenic therapy

The term “pro-angiogenic therapy” as recited above relates to a therapy which induces or enhances the process of angiogenesis systemically or topically in a subject. Preferably, said pro-angiogenic therapy comprises administration of an pro-angiogenic drug, preferably, selected from the group consisting of: VEGF, PlGF, endoglin, anti-Flt-1 antibodies and ALK5 modifiers.

The term “susceptible” as used herein means that a statistically significant portion of subjects identified by the method as being susceptible respond to the envisaged therapy by showing angiogenesis in the affected areas of the heart.

In a preferred embodiment of the aforementioned method, a decreased amount of PlGF and sFlt-1 and a increased amount of endoglin exclude a subject as being susceptible to a pro-angiogenic therapy.

The information whether these agents should be administered is provided if an elevated (lowered) level of PlGF which is indicative for anti-angiogenic processes is measured.

When a level of PlGF of >8 pg/ml, preferably >10 pg/ml, more preferably >12 pg/ml, in particular >15 pg/ml is reached, one or more of the above-cited drugs should be administered.

The information whether these agents should be administered can also be provided if an elevated level of endoglin and/or sFlt-1 which is indicative for angiogenic processes is measured.

When a level of endoglin of >4.8 pg/ml, preferably >4.4 pg/ml, more preferably >4.2 pg/ml is reached, one or more of the above-cited drugs should be administered. When a level of sFlt-1 of >142 pg/ml, more preferably ≧125 pg/ml, more preferably ≧110 pg/ml, in particular ≧90 pg/ml is reached, one or more of the above-cited drugs should be administered.

D) In general, troponin I and/or T, in particular troponin T, is indicative of an existing myocardial necrosis and the extent of the necrosis; in case no drop in the level of troponin T/I is observed, then this peptide indicates heart failure and/or vascular stenosis which can be treated by percutane coronary intervention.

The information whether these agents should be administered if an elevated level of troponin I and/or troponin T, in particular troponin T which is indicative for heart failure or vascular stenosis is measured.

The present invention further encompasses a device for diagnosing if a subject suffering from dilated cardiomyopathy is suffering from ischemic or non-ischemic dilated cardiomyopathy comprising:
a) means for determining the amounts of the following peptides:
   troponin or a variant thereof; and
   GDF-15 or a variant thereof;
   one or more angiogenic markers from the group PlGF or a variant thereof; endoglin or a variant thereof, and sFlt-1 or a variant thereof;
   optionally, a natriuretic peptide or a variant thereof;
   in a sample of a subject suffering from dilated cardiomyopathy; and
b) means for comparing the amounts determined in step a) with reference amounts, whereby it is to be diagnosed whether the subject suffers from either ischemic or non-ischemic dilated cardiomyopathy,
   whereby the device is adapted for carrying out the method of the present invention referred to above.

The term “device” as used herein relates to a system of means comprising at least the aforementioned means operatively linked to each other as to allow the prediction. Preferred means for determining the amount of a one of the aforementioned polypeptides as well as means for carrying out the comparison are disclosed above in connection with the method of the invention. How to link the means in an operating manner will depend on the type of means included into the device. For example, where means for automatically determining the amount of the peptides are applied, the data obtained by said automatically operating means can be processed by, e.g., a computer program in order to obtain the desired results. Preferably, the means are comprised by a single device in such a case. Said device may accordingly include an analyzing unit for the measurement of the amount of the peptides or polypeptides in an applied sample and a computer unit for processing the resulting data for the evaluation. The computer unit, preferably, comprises a database including the stored reference amounts or values thereof recited elsewhere in this specification as well as a computer-implemented algorithm for carrying out a comparison of the determined amounts for the polypeptides with the stored reference amounts of the database. Computer-implemented as used herein refers to a computer-readable program code tangibly included into the computer unit. Alternatively, where means such as test strips are used for determining the amount of the peptides or polypeptides, the means for comparison may comprise control strips or tables allocating the determined amount to a reference amount. The test strips are, preferably, coupled to a ligand which specifically binds to the peptides or polypeptides referred to herein. The strip or device, preferably, comprises means for detection of the binding of said peptides or polypeptides to the said ligand. Preferred means for detection are disclosed in connection with embodiments relating to the method of the invention above. In such a case, the means are operatively linked in that the user of the system brings together the result of the determination of the amount and the diagnostic or prognostic value thereof due to the instructions and interpretations given in a manual. The means may appear as separate devices in such an embodiment and are, preferably, packaged together as a kit. The person skilled in the art will realize how to link the means without further ado. Preferred devices are those which can be applied without the particular knowledge of a specialized clinician, e.g., test strips or electronic devices which merely require loading with a sample. The results may be given as output of raw data which need interpretation by the clinician. Preferably, the output of the device is, however, processed, i.e. evaluated, raw data the interpretation of which does not require a clinician. Further preferred devices comprise the analyzing units/devices (e.g., biosensors, arrays, solid supports coupled to ligands specifically recognizing the natriuretic peptide, Plasmon surface resonance devices, NMR spectrometers, mass-spectrometers etc.) and/or evaluation units/devices referred to above in accordance with the method of the invention.

Also, the present invention relates to a device for diagnosing and/or deciding which medication is to be applied in a subject suffering form after dilated cardiomyopathy comprising:
a) means for determining the amounts of the following peptides:
   troponin or a variant thereof; and
   GDF-15 or a variant thereof;
   one or more angiogenic markers from the group PlGF or a variant thereof; endoglin or a variant thereof, and sFlt-1 or a variant thereof;
   optionally, a natriuretic peptide or a variant thereof;
   in a sample of a subject suffering from dilated cardiomyopathy; and
b) means for comparing the amounts determined in step a) with reference amounts, whereby it is to be diagnosed whether the subject suffers from either ischemic or non-ischemic dilated cardiomyopathy,
   whereby the device is adapted for carrying out the method of the present invention referred to above.

Moreover, the present invention relates to a kit adapted for carrying out the method of the present invention referred to above comprising:
a) means for determining the amounts of the following peptides:
   troponin or a variant thereof; and
   GDF-15 or a variant thereof;
   one or more angiogenic markers from the group PlGF or a variant thereof; endoglin or a variant thereof, and sFlt-1 or a variant thereof;
   optionally, a natriuretic peptide or a variant thereof;
   in a sample of a subject suffering from dilated cardiomyopathy; and
b) means for comparing the amounts determined in step a) with reference amounts, whereby it is to be diagnosed whether the subject suffers from either ischemic or non-ischemic dilated cardiomyopathy,
   whereby the kit is adapted for carrying out the method of the present invention referred to above. Preferably, the kit comprises instructions for carrying out the said method of the present invention.

The term “kit” as used herein refers to a collection of the aforementioned means, preferably, provided in separately or within a single container. The container, also preferably, comprises instructions for carrying out the method of the present invention.

### Specific Embodiments

The following Examples shall merely illustrate the invention. They shall not be construed, whatsoever, to limit the scope of the invention.

A total of 114 patients with ischemic cardiomyopathy and 68 patients with non-ischemic cardiomyopathy were included into the study. All patients were assessed by echocardiography and had a left ventricular ejection fraction of less than 30%. In addition all patients received a coronary angiography, based on the results of this procedure patients were assigned to ischemic or non-ischemic cardiomyopathy,

In both tables, I stands for ischemic DCM, NI stands for non-ischemic DCM.

Table 1 shows the numerical values for the marker level for both patient groups.

TABLE 1
Biomarkers in Patients with Ischemic and Non-Ischemic Cardiomyopathy

| | NT-proBNP | | Hs-Troponin T | | PlGF | | sFlt-1 | |
|---|---|---|---|---|---|---|---|---|
| | [pg/ml] | | [pg/ml] | | [pg/ml] | | [pg/ml] | |
| | N = 114 | N = 68 | N = 114 | N = 68 | N = 114 | N = 68 | N = 114 | N = 68 |
| | I | NI | I | NI | I | NI | I | NI |
| | CMP | CMP | CMP | CMP | CMP | CMP | CMP | CMP |
| Median | 421 | 309 | 9.2 | 6.8 | 11.5 | 9.8 | 94 | 135 |
| 75th percentile | 1475 | 1488 | 25.5 | 15.2 | 15.8 | 13.3 | 115 | 177 |
| 95th percentile | 6771 | 16276 | 131.7 | 54.6 | 20.7 | 17.2 | 275 | 388 |
| 5th percentile | 32 | 13 | 1.0 | 1.0 | 7.0 | 7.0 | 55 | 83 |
| 25th percentile | 137 | 119 | 4.5 | 3.5 | 7.4 | 7.0 | 76 | 109 |

| | Endoglin | | GDF-15 | | NT-proBNP/GDF-15 | |
|---|---|---|---|---|---|---|
| | [ng/ml] | | [pg/ml] | | Ratio | |
| | N = 114 | N = 68 | N = 114 | N = 68 | N = 114 | N = 68 |
| | I | NI | I | NI | I | NI |
| | CMP | CMP | CMP | CMP | CMP | CMP |
| Median | 4.33 | 5.75 | 1205 | 2186 | 1.58 | 1.48 |
| 75th percentile | 4.88 | 6.93 | 1937 | 4107 | 5.01 | 3.78 |
| 95th percentile | 5.90 | 8.45 | 337 | 604 | 0.05 | 0.02 |
| 5th percentile | 3.34 | 2.94 | 598 | 842 | 0.19 | 0.13 |
| 25th percentile | 3.83 | 4.56 | 897 | 1220 | 0.47 | 0.33 |

Table 2 shows the correlation between NT-pro BNP and other diagnostic tests and the differences in correlation in ischemic and non-ischemic cardiomyopathy.

TABLE 2

| | Cardiomyopathy | |
|---|---|---|
| Correlation | Ischemic | Non-ischemic |
| Sensitive Troponin T vs. NT-proBNP | R² = 0.5280 | R² = 0.0818 |
| GDF-15 vs. NT-proBNP | R² = 0.5938 | R² = 0.3439 |
| Endoglin vs. NT-proBNP | R² = 0.0015 | R² = 0.0345 |
| PlGF vs. NT-proBNP | R² = 0.0128 | R² = 0.0060 |
| sFlt-1 (sVEGFR) vs. NT-proBNP | R² = 0.4556 | R² = 0.0126 |

What is claimed is:

1. A method for diagnosing if a subject suffering from dilated cardiomyopathy is suffering from ischemic or non-ischemic dilated cardiomyopathy, the method comprising:
determining in a sample from the subject an amount of a cardiac troponin, an amount of growth differentiation factor-15 (GDF-15), and an amount of an angiogenic marker selected from the group consisting of placental growth factor (PlGF), endoglin, and soluble Flt-1 (sFlt-1),
comparing the amounts determined with a reference amount of the cardiac troponin, GDF-15, and the angiogenic marker, respectively, wherein a diagnosis that the subject is suffering from non-ischemic dilated cardiomyopathy is indicated if the amounts of GDF-15, endoglin, and sFlt-1 determined are increased over the reference amounts and the amounts of cardiac troponin and PlGF are decreased over the reference amounts, and wherein a diagnosis that the subject is suffering from ischemic dilated cardiomyopathy is indicated if the amounts of PlGF and cardiac troponin determined are increased over the reference amounts and the amounts of GDF-15, sFlt-1 and endoglin are decreased over the reference amounts.

2. The method of claim 1, wherein the angiogenic marker is PlGF.

3. The method of claim 1, wherein the angiogenic marker is sFlt-1.

4. The method of claim 1, wherein the cardiac troponin is troponin T.

5. The method of claim 4, wherein an amount of troponin T larger than 0.008 ng/ml is indicative for an ischemic state.

6. The method of claim 4, wherein an amount of troponin T less than 0.008 ng/ml is indicative for a non-ischemic state.

7. The method of claim 1, further comprising determining an amount of a natriuretic peptide and comparing the amount determined with a reference amount of the natriuretic peptide wherein a diagnosis of myocardial dysfunction and/or heart failure if indicated if the amount of natriuretic peptide is greater than the reference amount.

8. The method of claim 7, wherein the natriuretic peptide is N-terminal pro-brain natriuretic peptide (NT-proBNP).

9. The method of claim 8, wherein an amount of NT-proBNP greater than 125 pg/ml is indicative for heart failure.

10. The method of claim 1, wherein a diagnosis that the subject is suffering from ischemic dilated cardiomyopathy is indicated if the amount of GDF-15 is less than 600 pg/ml, the amount of PlGF is greater than 11 pg/ml, the amount of endoglin is less than 4.2 ng/ml, or the amount of sFlt-1 is less than 120 pg/ml.

11. The method of claim 1, wherein a diagnosis that the subject is suffering from non-ischemic dilated cardiomyopathy is indicated if the amount of GDF-15 is greater than 1000 pg/ml, the amount of PlGF is less than 8 pg/ml, the amount of endoglin is greater than 4.8 ng/ml, or the amount of sFlt-1 is greater that 142 pg/ml.

12. A method for determining which medication is to be administered to a subject suffering from dilated cardiomyopathy, the method comprising
determining in a sample from the subject an amount of a cardiac troponin, an amount of growth differentiation factor-15 (GDF-15), and an amount of an angiogenic marker selected from the group consisting of placental growth factor (PlGF), endoglin, and soluble Flt-1 (sFlt-1),
comparing the amounts determined with a reference amount of the cardiac troponin, GDF-15, and the angiogenic marker, respectively, wherein a diagnosis that the subject is suffering from non-ischemic dilated cardiomyopathy is indicated if the amounts of GDF-15, endoglin, and sFlt-1 determined are increased over the reference amounts and the amounts of cardiac troponin and PlGF are decreased over the reference amounts, and wherein a diagnosis that the subject is suffering from ischemic dilated cardiomyopathy is indicated if the amounts of GDF-15, endoglin, and sFlt-1 determined are decreased over the reference amounts and the amounts of cardiac troponin and PlGF are increased over the reference amounts, and
wherein, when a diagnosis is made that the subject is suffering from ischemic dilated cardiomyopathy, it is determined that a medication is to be administered to the subject, wherein the medication is selected from the group consisting of statins, ACE inhibitors, angiotensin receptor antagonists, and aldosterone antagonists.

13. A device for diagnosing if a subject suffering from dilated cardiomyopathy is suffering from ischemic or non-ischemic dilated cardiomyopathy, the device comprising:
a means for determining in a sample from the subject an amount of a cardiac troponin, an amount of growth differentiation factor-15 (GDF-15), and an amount of an angiogenic marker selected from the group consisting of placental growth factor (PlGF), endoglin, and soluble Flt-1 (sFlt-1), and
a means for comparing the amounts determined with reference amounts of the cardiac troponin, GDF-15, and the angiogenic marker, respectively.

14. A device for determining which medication is to be administered to a subject suffering from dilated cardiomyopathy, the device comprising:
a means for determining in a sample from the subject an amount of a cardiac troponin, an amount of growth differentiation factor-15 (GDF-15), and an amount of an angiogenic marker selected from the group consisting of placental growth factor (PlGF), endoglin, and soluble Flt-1 (sFlt-1), and
a means for comparing the amounts determined with reference amounts of the cardiac troponin, GDF-15, and the angiogenic marker, respectively, whereby a diagnosis of ischemic or non-ischemic dilated cardiomyopathy is enabled and thereby a determination of medication.

15. A kit for diagnosing if a subject suffering from dilated cardiomyopathy is suffering from ischemic or non-ischemic dilated cardiomyopathy according to the method of claim 1, the kit comprising:
reagent means for specifically determining in a sample from a subject an amount of a cardiac troponin, an amount of growth differentiation factor-15 (GDF-15), and an amount of an angiogenic marker selected from the group consisting of placental growth factor (PlGF), endoglin, and soluble Flt-1 (sFlt-1), and
instructions for comparing the amounts determined with reference amounts and diagnosing whether the subject suffers from ischemic or non-ischemic dilated cardiomyopathy.
